(19) United States
(12) Patent Application Publication
(10) Pub. No.: US 2014/0276243 A1
(43) Pub. Date: Sep. 18, 2014

(54) BEHAVIORAL RISK ANALYZER AND APPLICATION THAT ESTIMATES THE RISK OF PERFORMING UNDESIRED BEHAVIOR

(71) Applicant: Koninklijke Philips N.V., Eindhoven (NL)

(72) Inventors: SASKIA VAN DANTZIG, UTRECHT (NL); MAURO BARBIERI, EINDHOVEN (NL); TESS SPEELPENNING, EINDHOVEN (NL); MONIQUE HENDRIKS, EINDHOVEN (NL)

(73) Assignee: KONINKLIJKE PHILIPS N.V., EINDHOVEN (NL)

(21) Appl. No.: 14/178,783

(22) Filed: Feb. 12, 2014

Related U.S. Application Data
(60) Provisional application No. 61/789,323, filed on Mar. 15, 2013.

(51) Int. Cl. A61B 5/11; G09B 19/00

(52) U.S. Cl. CPC A61B 5/1118; G09B 19/00
USPC 600/595; 434/236

(57) ABSTRACT

A method for coaching a subject, including receiving data corresponding to at least one of a desired behavior of the subject or an undesired behavior of the subject, receiving a level of available self control of the subject via an input source, calculating a level of required self control needed to perform the desired behavior or suppress the undesired behavior, analyzing a risk based on the level of available self control and the level of required self control, where the risk is associated with predicting whether the subject will perform the undesired behavior or not perform the desired behavior, and intervening when the risk analyzed is above a particular threshold.

### TECHNICAL FIELD

The present disclosure relates to the field of promoting a healthier lifestyle to a subject and in particular to a system, method, and computer readable medium for estimating a risk associated with performing an undesired behavior and/or not performing a desired behavior.

### BACKGROUND

Changing behavior and maintaining a novel behavior is a very demanding process, which requires a large amount of self-control. The amount of self-control of an individual depends on many factors and fluctuates throughout the day. In addition, some situations require more self-control than others.

Each New Year's Day, for example, millions of people vow to lead a healthier life. Health-related behaviors such as losing weight, eating more healthily, exercising more and quitting smoking invariably appear in the top-ten lists of New Year's resolutions. Unfortunately, although most people believe that they will be successful, only a few of them actually succeed in maintaining their new behaviors. One of the reasons for this low success rate is that changing behavior is a very demanding process, which requires a large amount of willpower. People who are in a behavioral change trajectory frequently have to suppress habitual responses or replace these habits with alternative, healthier, actions.

For example, consider an individual, who attempts to incorporate more physical activity into his daily life. The individual has resolved to cycle to work every day, to take a lunch walk frequently and to perform sports twice a week. Suppressing his old habits (taking the car, spending an evening on the couch) and replacing it by healthier alternatives (taking the bike, going out for a run) requires a large amount of self-control. The individual especially needs support at those moments when he is most likely to lapse into his old behavior. In other words, when the individual's self-control is insufficient to face the demands of the situation. For example, suppose that the individual wakes up late after a bad night's sleep because his young baby woke up multiple times during the night. He does not have the time to eat a proper breakfast. It is cold outside, and rain is forecasted for the day. Together, these factors make that the individual cannot resist the temptation of taking the car to work instead of the bike.

The individual could be helped by a coaching system that intervenes before or during such “high-risk situations,” suggesting ways to increase his level of self-control, to avoid the situation, or to replace his behavior by another behavior. Such a coaching system should be able to predict when these high-risk situations occur.

### SUMMARY

This challenge is addressed by the present disclosure which includes a method to compute the moment-to-moment risk of performing unhealthy or undesired behavior, based on the difference between the available amount of self-control and the required amount of self-control of a particular individual. The output from this risk analysis is used as input for a coaching system.

The coaching and intervention can be focused on those moments that really matter. As a result, users are not bothered by the system during moments when they do not need any support. Although current coaching systems try to define opportune moments for interaction with the user, they define those moments based on other aspects of the situation (e.g., time, location and agenda). No existing system has yet tried to select moments based on a risk assessment of the behavior under consideration.

According to an aspect of the present disclosure a method for coaching a subject is provided including receiving data corresponding to at least one of a desired behavior of the subject or an undesired behavior of the subject, calculating a level of available self control of the subject based on input from various sources, calculating a level of required self control needed to perform the desired behavior or suppress the undesired behavior, analyzing a risk based on the level of available self control and the level of required self control, wherein the risk is associated with predicting whether the subject will perform the undesired behavior or not perform the desired behavior, and intervening when the risk analyzed is above a particular threshold. The data received may be extracted from a calendar. The level of available self control may be extracted from images of food consumed or via a food log. The input source may take the form of an activity monitor with accelerometer features and/or heart rate monitoring features. The intervention may include delivering a notification to the subject suggesting that the subject perform an action or refrain from performing an action to lower the risk to a value below the threshold.

According to another aspect of the present disclosure, a system for coaching a subject is provided, including a processor, and a memory storing instructions, executable by the processor, wherein the instructions when executed by the processor cause the system to: receive data corresponding to at least one of a desired behavior of the subject or an undesired behavior of the subject, calculate a level of available self control of the subject based on several input sources, calculate a level of required self control needed to perform the desired behavior or suppress the undesired behavior, analyze a risk based on the level of available self control and the level of required self control, wherein the risk is associated with predicting whether the subject will perform the undesired behavior or not perform the desired behavior; and intervene when the risk analyzed is above a particular threshold. The data received may be extracted from a calendar. The level of available self control may be extracted from images of food consumed or via a food log. The input source may take the form of an activity monitor with accelerometer features and/or heart rate monitoring features. The intervention may include delivering a notification to the subject suggesting that the subject perform an action or refrain from performing an action to lower the risk to a value below the threshold.

According to another aspect of the present disclosure, an apparatus is provided including a receiving unit configured to receive data corresponding to at least one of a desired behavior of a subject or an undesired behavior of a subject, a calculating unit configured to calculate a level of available self control of the subject and a level of required self control needed to perform the desired behavior or suppress the undesired behavior, a risk assessment unit configured to analyze a risk based on the level of available self control and the level of required self control, wherein the risk is associated with predicting whether the subject will perform the undesired behavior or not perform the desired behavior, and an intervention unit configured to intervene when the risk analyzed by the risk assessment unit is above a particular threshold. The data received may be extracted from a calendar. The level of available self control may be extracted from images of food consumed or via a food log. The input source may take the form of an activity monitor with accelerometer features and/or heart rate monitoring features. The intervention may include delivering a notification to the subject suggesting that the subject perform an action or refrain from performing an action to lower the risk to a value below the threshold.

According to another aspect of the present disclosure a non-transitory computer readable storage medium is provided, storing a program, which when executed by a computer, causes the computer to a perform a method for coaching a subject including the steps of receiving data corresponding to at least one of a desired behavior of the subject or an undesired behavior of the subject, receiving a level of available self control of the subject via an input source, calculating a level of required self control needed to perform the desired behavior or suppress the undesired behavior, analyzing a risk based on the level of available self control and the level of required self control, wherein the risk is associated with predicting whether the subject will perform the undesired behavior or not perform the desired behavior, and intervening when the risk analyzed is above a particular threshold. The data received may be extracted from a calendar. The level of available self control may be extracted from images of food consumed or via a food log. The input source may take the form of an activity monitor with accelerometer features. The intervention may include delivering a notification to the subject suggesting that the subject perform an action or refrain from performing an action to lower the risk to a value below the threshold.

### BRIEF DESCRIPTION OF THE FIGURES

The aspects of the present disclosure may be better understood with reference to the following figures. The components in the figures are not necessarily to scale, emphasis instead being placed on the clearly illustrating the principles of the disclosure. Moreover, in the figures, like reference numerals designate corresponding parts throughout the several views.

In the figures:

FIG. 1 shows a schematic representation of components of the system of the present disclosure and illustrates the cooperation of these components in accordance with an embodiment of the present disclosure;

FIG. 2 shows a schematic representation of the components of a computing resource of the system of FIG. 1;

FIG. 3 shows a chart illustrating an example of fluctuation of levels of available self-control throughout the day;

FIG. 4 shows a chart illustrating an example of the levels of available self-control since awakening;

FIG. 5 shows the level of required self-control as a function of the variance of the past activity and the delta between planned and past activity;

FIG. 6 shows a schematic representation of the system of the present disclosure in accordance with an embodiment of the present disclosure;

FIG. 7 shows a schematic representation of the system of the present disclosure in accordance with another embodiment of the present disclosure; and

FIG. 8 shows a flow chart illustrating a method for coaching a subject using risk analysis in accordance with an embodiment of the present disclosure.

### DETAILED DESCRIPTION OF EMBODIMENTS

The present disclosure describes various embodiments of systems, methods, and devices for estimating a risk associated with performing an undesired behavior and/or not performing a desired behavior and intervening when the risk is above a threshold.

As represented in FIG. 1, shown is a system 100 according to various embodiments. The system 100 includes a computing resource 101, client device 102a, and a network 104 which may be any type of communication layer. The computing resource 101 includes a processor 107c and a memory 108c that stores an application. The computing resource 101 may be a server, computer, or another device providing computing capability. In some embodiments, the computing resource 101 includes a plurality of computing resources that are arranged, for example, in one or more server banks, computer banks or other arrangements. Further, in some embodiments, the computing resource 101 includes a cloud computing resource, a grid computing resource, or any other distributed computing arrangement. For purposes of convenience, a computing resource is referred to herein in the singular, but it is understood that a plurality of computing resources may be employed in the various arrangements described above instead. Although application 110 is shown and described herein as being a component of computing resource 101, it is also envisioned that application 110 may be a component of client device 102a.

A client device 102 (e.g., denoted as client device 102a) is representative of a plurality of client devices that may be coupled to the network 104. In the embodiment illustrated in FIG. 1, the client device 102a is associated with a subject (i.e., a user, client, coachee). The client device 102a may be configured to communicate with an activity monitor 105, which will be discussed in further detail below. Additionally, or alternatively, the activity monitor 105 may be configured to communicate with the computing resource 101 over the network 104 without a client device 102 as an intermediary. Client devices 102 may be configured to receive data from activity monitor 105, or otherwise transmit data between activity monitor 105, client devices 102, and computing resource 101, as will be described in further detail below. Although activity monitor 105 is shown and described as being a separate component, unit, or element, from client device 102, it is also envisioned that client device 102, in particular client device 102a, may be configured to perform all of the functions of activity monitor 105. In some embodiments, the activity monitor 105 may be included in, or removably attached to, a watch, glasses, headphones, or other electronic device that is worn by a user.

A client device 102 may include, for example, a processor-based system such as a computer system. Such a computer system may be embodied in the form of a desktop computer, a laptop computer, a personal digital assistant, a mobile device, a cellular telephone, a smart phone, a set-top box, a music player, a web pad, a tablet computer system, a gaming console, or other devices with like capability. The client device 102 may be configured to execute various applications such as a browser and/or other applications. When executed in a client device 102, the browser may render network pages, such as web pages, on a display device and may perform other functions. The browser may be executed in a client device 102 for example, to access, render, or display network pages, such as web pages, or other network content served up by the computing resource 101 and/or other servers. The client device 102 may be configured to execute applications other than a browser such as, for example, email applications, instant message applications, mobile applications, and/or other applications.

The network 104 includes, for example, the Internet, intranets, extranets, wired networks, wireless networks, wide area networks (WANs), local area networks (LANs), or other suitable networks, etc., or any combination of two or more such networks.

The computing resource 101 and client devices 102 each respectively include a processor 107 and a memory 108. In the embodiment illustrated in FIG. 1, the client device 102a includes a processor 107a and a memory 108a. Further, the computing resource 101 includes a processor 107c and a memory 108c. In some embodiments, the computing resource 101 and client device 102 may include more than one processor 107 and more than one memory 108. For purposes of convenience, the processor 107 and memory 108 are referred to herein in the singular, but it is understood that a plurality of processors 107 and/or a plurality of memories 108 may be employed by a computing resource 101 or a client device 102.

Processor 107 is configured to process any of the steps or functions of computing resource 101 and/or system 100, and/or any of the modules, units, or components thereof. The term processor, as used herein, may be any type of controller or processor, and may be embodied as one or more controllers or processors adapted to perform the functionality discussed herein. Additionally, as the term processor is used herein, a processor may include use of a single integrated circuit (IC), or may include use of a plurality of integrated circuits or other components connected, arranged or grouped together, such as controllers, microprocessors, digital signal processors, parallel processors, multiple core processors, custom ICs, application specific integrated circuits, field programmable gate arrays, adaptive computing ICs, associated memory, such as and without limitation, RAM, DRAM and ROM, and other ICs and components.

A memory 108 may include both volatile and/or nonvolatile memory and data storage components. Volatile components are those that do not retain data values upon loss of power. Nonvolatile components are those that retain data upon a loss of power. Thus, the memory may include, for example, random access memory (RAM), read-only memory (ROM), hard disk drives, solid-state drives, USB flash drives, memory cards accessed via a memory card reader, floppy disks accessed via an associated floppy disk drive, optical discs accessed via an optical disc drive, magnetic tapes accessed via an appropriate tape drive, and/or other memory components, or a combination of any two or more of these memory components. In addition, the RAM may include, for example, static random access memory (SRAM), dynamic random access memory (DRAM), or magnetic random access memory (MRAM) and other such devices. The ROM may include, for example, a programmable read-only memory (PROM), an erasable programmable read-only memory (EPROM), an electrically erasable programmable read-only memory (EEPROM), another like memory device. A memory 108 is a computer readable medium.

Further, a memory 108 may store instructions that are executable by the processor 107. For example, the memory 108c of the computing resource 101 stores instructions for the application 110 for promoting a healthier lifestyle of a subject and coaching a subject using risk analysis. The term subject designates the user associated with client device 102a, and this user is the coachee (i.e., the person who is coached by the system 100 and/or the coach). This person may also be designated as customer, client, and/or subject in the present text.

Turning now to FIG. 2, shown is a detailed view of the computing resource 101 of FIG. 1. Computing resource 101 includes processor 107c, memory 108c, receiving unit 111, calculating unit 113, risk analyzing unit 115, and intervention unit 117, the functions of each of which will be described in further detail below with reference to FIGS. 6-8. The receiving unit 111 is configured to receive data corresponding to at least one of a desired behavior of a subject or an undesired behavior of a subject (also referred to herein as intention data). The desired behavior and undesired behavior may be data which is input into system 100, or otherwise specified, by a user. The desired behavior can include, for example, the desire of the subject to ride a bicycle after work. The undesired behavior can include, for example, smoking a cigarette or eating a particular food that the subject does not want to consume. The receiving unit 111 may receive the data, for example and without limitation, from a subject that inputs the data into the system 100.

The calculating unit 113 is configured to calculate the level of available self-control of the subject and the level of self control-required to perform the desired behavior or refrain from performing the undesired behavior. The risk analyzing unit 115 is configured to assess, or otherwise analyze, the risk of whether the subject will perform the desired behavior and/or not perform the undesired behavior based on the level of available self-control and the level of required self-control. The intervention unit 117 is configured to intervene when the risk analyzed by the risk analyzing unit 115 exceeds a particular threshold. The intervention unit 117 may intervene in several different ways, for example and without limitation, by sending a notification to the subject. Such intervention can consist of for example, actionable advice how to replenish self-control before facing the high-risk situation (e.g., by taking some high-glucose food, taking some rest, improving one's mood), actionable advice how to lower the self-control demand (e.g., by avoiding particular situations, performing alternative actions), activating a peer, who could provide moral support.

Turning now to FIGS. 3 and 4, the level of available self-control will now be described, and will be described in further detail with reference to the particular embodiments of the present disclosure. The available level of self-control is estimated based on several sources of input. Information is gathered by dedicated devices (e.g., an activity monitor, sleep monitor, heart rate monitor, etc.), through manual user input (e.g., questionnaires, experience samples, etc.), or derived from other sources of information about one's context (e.g., digital calendar, social networks, etc.).

The available level of self-control may be derived, for example and without limitation, from the following aspects: a. ‘Stable’ personal characteristics (e.g., assessed by a questionnaire), defining the base level of self-control; b. Amount of training, for example, the base-level of self-control can be increased through training (as in muscle training, exerting self-control depletes the resource at the short term, but leads to a higher base level of self-control at the long term); c. Data about amount of sleep and sleep quality (for example sleep may restore the level of self-control); d. Data about timing and type of food intake, predicting the blood glucose level (for example, low levels of blood glucose are associated with low level of self-control); e. Data about any other factors that may deplete or restore self-control (e.g., stressful situations, demanding tasks, mood, etc.); f. heart rate variability (HRV) data which could be a marker of the amount of exerted or available self-control; and g skin conductance which may be used to detect mood behavior of a subject.

FIG. 3 illustrates an example of how the level of available self-control may fluctuate across the day. As shown, the level of self-control gradually depletes over time throughout the day. The depletion rate depends on the demands of the tasks that have to be performed. Additionally, self-control may be restored by certain activities or actions such as sleep, rest, and food intake. The grey areas of the chart show activities such as sleep and food intake, where the self-control is restored and thus rises.

FIG. 4 shows a chart illustrating an example of the levels of available self-control since awakening.

The level of available self-control is compared to the level of required self-control. This refers to the level of self-control needed to perform a desired behavior (e.g., performing physical activity) or suppress an undesired behavior (e.g., smoking a cigarette) in a particular situation. The required level of self-control depends on various characteristics of the situation and the behavior under consideration, for example: 1) Is the behavior habitual? Executing a habitual behavior is likely to require little self-control. In contrast, suppressing or overriding a habitual behavior is likely to require much self-control; 2) Are the means to execute a behavior available? (e.g., an individual does not need much self-control to take his bike if there is no alternative available, e.g., because his car is broken); 3) Is the behavior in line with that of one's peers? We assume that more self-control is needed to deviate from the behavior of peers (e.g., an individual needs more self-control if he is the only one taking a lunch walk than when it is common practice among his colleagues to take lunch walks); 4) Does the person expect to need much self-control at a later moment (e.g., a student planning to study all night for a difficult exam)? In such a case, the person might ‘decide’ to save some self-control by giving in to an urge (e.g., smoking a cigarette). This process is called self-control conservation.

Based on the balance between available and required self-control, a moment-to-moment risk analysis is made by the risk analyzing unit 115 (FIG. 2), predicting the likelihood that the user will perform a particular undesired behavior (or fail to perform a desired behavior), as will be described in further detail below. If the level of available self-control is lower than the level of required self-control, there is a high risk of performing the undesired behavior (or refraining from the desired behavior).

For example, consider the following situations: 1. an individual wakes up late after a bad night's sleep, because his young baby woke up multiple times during the night. He does not have the time to eat a proper breakfast, it is cold outside, and rain is forecasted for the day. Because he slept badly and has not eaten well, the individual's self-control is too low to face the barriers of the situation (the cold and rain). As a result, the individual cannot resist the temptation of taking the car instead of the bike. 2. At lunchtime, the individual debates whether he will take a lunch walk. This morning he had a tough and demanding meeting, which has depleted his level of self-control. He has not eaten anything since breakfast, so his glucose level is low. When his colleagues invite him to join them for lunch in the canteen, he decides to join them, despite his resolution to take lunch walks more often. Based on the outcome of the risk analysis, the system 100 may decide to intervene before or during high-risk situation.

Turning now to FIGS. 6 and 7, particular embodiments of system 100 will now be described with particular detail. In an embodiment the behavioral risk analyzer is embedded in a physical activity coaching service, running on a smart phone. The coaching service aims to support users in becoming more active during a multiple-week activity program. At the start of the program, the user indicates his activity plans for the upcoming period. For example, he might plan to cycle to work daily, to take lunch walks on Monday and Thursday, and to go for a run on Tuesday evening and Saturday morning.

System 100, in particular receiving unit 111, receives continuous input about the user's physical activity from an activity monitor. Calculating unit 113 computes the available amount of self-control and the required amount of self-control. Based on the balance between available and required amounts of self-control, the risk analyzing unit 115 provides as output a moment-to-moment risk assessment of the current situation or a future situation. Based on this risk assessment, the intervention unit 117 determines if a threshold has been exceeded and the most suitable intervention to be executed before or during a high-risk moment.

In particular, suppose that scₐᵥₐᵢₗ(t, d, i) is the available level of self-control of individual i on day d at time t, and sc_{req}(t, d, i) is the required level of self-control of individual i on day d at time t. Based on the difference between scₐᵥₐᵢₗ(t, d, i) and sc_{req}(t, d, i), the risk r(t, d, i) can be computed by risk analyzing unit 115. The risk can be given with a certain chance and a confidence level. When r(t, d, i) exceeds a given threshold, the situation is assessed as high-risk, and an intervention can be triggered by intervention unit 117.

In particular, in an embodiment, the calculating unit 113, calculates the level of available self-control in the following manner:

Available level of self-control: Suppose that scₐᵥₐᵢₗ(t, d, i) is the available level of self-control of individual i at time t on day d. Several factors influence scₐᵥₐᵢₗ(t, d, i); some deplete the level of self-control, whereas others restore it. In this embodiment, the level of available self-control will be determined on the basis of three factors: the baseline level of self-control, the amount of sleep, and the time of the day, all of which are detailed below.

Individual differences in depletion rate: We assume that people vary in their trait level of self-control. That is, some people have more self-control than others. These individual differences can be modeled as different baseline self-control levels. The baseline level can be assessed by means of a questionnaire. Since it is assumed that baseline self-control is a more or less stable personality trait, this questionnaire needs to be filled in only once, when the user starts using the system. With respect to modeling the baseline level in estimating the available self-control, there are two possibilities. The first possibility is that people with more self-control have a higher overall level of available self-control. The second possibility is that people with more self-control are less affected by depleting factors. In this embodiment we will adhere to the second option.

Let depletionfactor(i) be a factor that influences the depletion rate of individual i. The value is determined by the outcome of an initial questionnaire score(i), weighted by a factor w1.

depletionfactor(i)=w₁·score(i)

Sleep: Sleep restores self-control. Sleep duration and quality could be measured by using an activity monitor 105 or a separate sleep monitor. Alternatively, the activity monitor 105 can provide an estimate of the sleep duration. Let scₐᵥₐᵢₗ₀ (d,i) be the level of self-control during a day. Let sleepduration(d) be the sleep duration of the night preceding day d. Let maxduration(i) be the maximum sleep duration of individual i. The value can be based on manual user input, or derived from historical data. scₐᵥₐᵢₗ₀(d,i) is computed using a linear function of the sleep duration divided by the maximum duration:

$${sc}_{avail 0} ( d , i ) = \frac{min [ sleepduration ( d , i ) , maxduration ( i ) ]}{maxduration ( i )}$$

Time since awakening: It is assumed that the available level of self-control gradually drops throughout the day, as a (linear) function of time. (See FIGS. 3 and 4). Time since awakening can be determined by combining the clock time with the wake-up time (derived from the activity monitor 105). Let scₐᵥₐᵢₗ(t, d, i) denote the available level of self-control of individual i in on day d at time t. Let t wakeup(d, denote the wake-up time of individual i on day d. The amount of self-control is depleted over time, at a rate that is determined by weight factor w2 and the individual's depletion factor(i).

$${sc}_{avail} ( t , d , i ) = {sc}_{av 0} ( d , i ) \cdot \frac{{w}_{2}}{t - {t}_{wakeup} ( d ) + {w}_{2}} \cdot depletionfactor ( i )$$

An example of the resulting curve is illustrated in FIG. 4.

The calculating unit 113 calculates the level of required self-control in the following manner and considering the following factors:

Required level of self-control: Let sd_{req}(t, d, i) be the estimated necessary level of self-control of individual i on day d and time t. Several factors influence sd_{req}(t, d, i); some lower the required self-control, whereas others increase it as detailed below.

Habit strength (with reference back to FIG. 5): Executing a habitual behavior requires little self-control. In contrast, suppressing or overriding a habitual behavior requires much self-control. Whether a particular behavior is habitual can be assessed from historical data, by looking at the co-variation between situations and behaviors. If the user has frequently and invariably executed the same behavior in the same situation, this behavior is strongly habitual. Instead, if a behavior is executed at random moments and times, it is less habitual. The amount of required self-control relates to the habitual strength of the behavior. Adhering to a desired behavior requires less self-control when this behavior has become a habit. Thus, someone attempting to exercise twice a week will find it less difficult to maintain this behavior if he always exercises on the same days than if he exercises on different days each week. Similarly, overriding an undesired behavior requires more self-control when this behavior is strongly habitual. In the case of physical activity, the habit strength can be derived from historical activity data. The planned activity on a certain day and time (e.g., take a lunch walk on Monday at noon) is compared with the activity that was realized at similar moments in the past (all earlier Mondays at noon).

Let act_{planned}(t, d, i) be the planned activity level of individual i at time t on day d. Let actₚₐₛₜ(t, d, i) be the average of the past activity during previous similar moments (same day and time). This average is calculated over a past period of several weeks (e.g. 1 month) or since the first time that the user has used the system. An arithmetic average can be used, or alternatively a weighted average in which more recent weeks are weighted heavier than more remote weeks.

Let SDₚₐₛₜ be the normalized standard deviation of actₚₐₛₜ(t, d, i). SDₚₐₛₜ varies between 0 and 1. Let delta_{act}(t, d, i) be the normalized difference between act_{planned}(t, d, i) and actₚₐₛₜ(t, d, i). delta_{act}(t, d, i) varies between 0 and 1. We assume that the required level of self-control depends on the delta between planned and realized activity and on the variance of the realized activity. If the delta is small and the variance is small, the planned behavior is similar to a habitual behavior (thus, the required self-control is low). If the delta is small and the variance is large, the planned behavior is similar to behavior that has been performed previously, but inconsistently (thus, the required level of self-control is intermediate). If the delta is large and the variance is small, the planned behavior deviates strongly from a habitual behavior (thus, the required level of self-control is high). If the delta is large and the variance is large, the planned behavior deviates from behavior that has been performed in the past. However, since the previous behavior was not performed consistently, the required level of self-control is intermediate.

This can be translated into the following function (where c1 is a constant between 0 and 1, and w3 has a value between 1/(0.5+|0.5−C₁|):

sc_{req}(t,d,i)=delta_{act}+(C₁−delta_{act}·w₃)·SDₚₐₛₜ

FIG. 5 illustrates the level of required self-control as a function of the variance of the past activity and the delta between planned and past activity.

The risk analyzing unit 115 uses the levels of available self-control and required self-control and determines whether the difference between the level of required self control and the level of available self-control exceeds a threshold. Additionally, these functions may be performed by intervention unit 117.

The intervention unit 117 determines when and how to intervene in the following manner:

The risk assessment analyzed by the risk assessment unit 115 can be used to trigger an intervention before or during a high-risk situation. There are different possibilities, depending on the lead time (time between assessment and actual high-risk situation) and on the target behavior. If there is much time (long lead time), the intervention unit 117 may suggest ways to avoid the situation. It can also suggest ways to ensure that self-control is sufficient for the target behavior. If there is not much time left (short lead time) and the situation cannot be avoided, the intervention unit 117 may suggest quick ways to replenish self-control (e.g., by glucose intake).

Examples of the two possibilities are sketched below.

Long lead time (24 hrs): The risk analyzing unit 115 predicts a high-risk situation tomorrow evening. The user has planned to exercise, but he will have a very busy day at work, and have no time to eat properly before his exercise. The system 100, in particular intervention engine 117, could warn him, and suggest ways to replenish his self-control (by taking enough sleep and food) or to avoid depletion (e.g., by rescheduling some of his tasks for tomorrow).

Short lead time (1 hr): The risk analyzing unit 115 predicts a high-risk situation in the next hour. The user has planned to take a lunch walk, but he has depleted his self-control during a long and demanding meeting. The system 100, in particular intervention unit 117, could suggest that he takes a small snack to increase his glucose level. Alternatively, it could suggest asking a colleague to join for the walk.

The system 100 can be applied in several domains. It can also be extended to include additional (domain-specific) factors that influence the available and required levels of self-control. Information about these factors is gathered through various means for example; via dedicated devices (e.g., activity monitor 105, sleep monitor), through manual user input on the smart phone (e.g., questionnaire, experience sampling), or derived from other sources (e.g., digital calendar, social network). The more information is available, the more accurate the estimated levels of self-control. However, not all information will be available at all times. If information about a certain factor is lacking, an assumption can be made based on historical data or based on default values. The confidence interval of the resulting estimate may then be increased, indicating that the estimate is uncertain. Several additional factors could be included to determine the available and required level of self-control, as described below.

Factors that influence the available level of self-control: Glucose level: Low levels of glucose are associated with low self-control. Glucose can be measured directly, but only by taking a blood sample. This may be too obtrusive. Alternatively, glucose level could be derived from the time and type of food intake. Information about time and type of food intake could be gathered in different ways for example and without limitation: The user enters the information manually on his smart phone; The user takes a picture of the food, which is analyzed by means of existing image recognition solutions.

To compute the blood glucose level, existing computer models can be used. The function to determine the available level of self-control based on the glucose level could be a linear transformation.

scₐᵥₐᵢₗ(t,d,i)=w₅·glucose(t,d,i)

Demanding tasks: Performing demanding tasks depletes self-control. To estimate the depletion of self-control on a particular day, the user's behavior on that day could be compared with his past behavior on similar days. The assumption is that deviating from a regular behavior is demanding. The function is similar to the one used to determine the required level of self-control. Let behavior_{current} (t, d, i) be the current behavior of individual i, on day d and time t. Let behaviorₚₐₛₜ (t, d, i) be the average past behavior of individual i, on similar weekdays and times. Let delta_{behavior}(t, d, i) be the normalized difference between behavior_{current}(t, d, i) and behaviorₚₐₛₜ(t, d, i). delta_{behavior}(t, d, i) varies between 0 and 1. Let SDₚₐₛₜ(t,d,i) be the normalized standard deviation of the past behavior, ranging between 0 and 1 (where c1 is a constant with a value between 0 and 1, and w3 is a constant with a value between 0 and 1/(0.5+|0.5−C₁|).

sc_{depletion}(t,d,i)=delta_{behavior}(t,i,d)+[C₁−delta_{behavior}(t,i,d)·w₃]·SDₚₐₛₜ(t,i,d)

Alternatively, to estimate the demands imposed on the user during a working day, information from his or her digital calendar could be used. Although it is difficult to assess how demanding each task is, various markers derived from the calendar could be used an indicator for the amount of required self-control. A possible marker could be the number of task switches during a day. Additional information about the imposed demand could be gathered from e.g., computer activity or by asking the user to provide a rating at various moments during the working day (experience sampling).

Factors that influence required self-control: Peer behavior: We assume that more self-control is needed to deviate from the behavior of one's peers. For example, if many people at a party smoke, the user will need more self-control to refrain from smoking Reversely, if many colleagues take a walk during lunchtime, the user will need less self-control to take a lunch walk as well. Information about peer behavior could be assessed in various ways for example: through a user questionnaire; derived from social network information; if the peers use the same system, derived from their user data. The required amount of self-control could be a function of the number of peers performing a particular behavior, their presence in the same situation, and the strength of their connection to the user.

Possibility to execute the desired/undesired behavior: Are the means to execute a behavior available? If it is impossible to execute a particular undesired behavior, no self-control is needed to override the behavior. This factor could be quite simple to measure in some domains, but difficult in others.

Anticipated need of self-control: Does the person expect to need much self-control at a later moment (e.g., a student planning to study all night for a difficult exam)? In that case, the person might ‘decide’ to save some self-control by giving in to an urge (e.g., smoking a cigarette). This process is called self-control conservation. Information about anticipated need of self-control can be derived from the digital calendar.

Detectability: When it is suspected that a particular coaching program uses the methods and systems of the present disclosure, the methods and systems of the present disclosure can be detected by testing the coaching program; one can take part in the program, perform specific behavior and check the timeliness and type of intervention.

An additional factor may be how much the target behavior (or its immediate outcome) is enjoyed. It requires more self-control to execute an unenjoyable behavior (e.g., performing heavy physical exercise). Reversely, it requires more self-control to refrain from an enjoyable behavior (e.g., drinking alcohol, smoking a cigarette). How much users enjoy various activities could be measured by means of questionnaires or through experience sampling.

Turning now to FIG. 8, a method for coaching a subject will now be described with particular detail as method 800. It is envisioned that method 800 may be implemented by system 100 and any of the components therein.

Method 800 begins with step 801 where receiving unit 111 receives intention data of a subject. The intention data may be data corresponding to desired or undesired behavior of the subject. In one embodiment, this data is extracted from a user's calendar. In another embodiment, the intention data is input by the user.

Subsequent to receiving the data in step 801, in step 803 the receiving unit 111 receives, or the calculating unit 113 otherwise calculates 113, data corresponding to the level of available self control of the subject via one or more input sources described above. In step 805, the calculating unit 113 proceeds to calculate the level of required self control necessary to perform the desired behavior or suppress the undesired behavior received in step 801. Once the levels are calculated in steps 803 and 805, method 800 then proceeds to step 807.

In step 807, risk analyzing unit 115 analyzes the risk, predicting the likelihood that the user will perform the undesired behavior (or fail to perform a desired behavior) received in step 801. If the level of available self-control is lower than the level of required self-control, there is a high risk of performing the undesired behavior (or refraining from the desired behavior). Once the risk is analyzed and determined in step 807, method 800 proceeds to step 809 where it is determined if the risk is above a particular threshold.

If the risk is not above the threshold (NO in step 809), then method 800 reverts back to step 801. Alternatively, if the risk is above the threshold (YES in step 809), then method 800 proceeds to step 811. In step 811 intervention engine 117 proceeds to intervene in any of the manners described above.

Although, the above-described embodiments have been described as being applicable to coaching and promoting a healthy lifestyle in a subject, it is envisioned that any of the above-described embodiments may be implemented in any system and may be used by any individuals not described above, for any purpose other that those described above.

Many modifications and other embodiments of the systems, methods, and devices of the present disclosure will come to mind to one skilled in the art having the benefit of the teachings presented in the foregoing descriptions and the associated drawings. Therefore, it is to be understood that the embodiments of the present disclosure are not to be limited to the specific embodiments disclosed and that modifications and other embodiments are intended to be included within the scope of the appended claims. Although specific terms are employed herein, they are used in a generic and descriptive sense only and not for purposes of limitation.

1. A method for coaching a subject, comprising:
receiving data corresponding to at least one of a desired behavior of the subject or an undesired behavior of the subject;
receiving a level of available self control of the subject via an input source;
calculating a level of required self control needed to perform the desired behavior or suppress the undesired behavior;
analyzing a risk based on the level of available self control and the level of required self control, wherein the risk is associated with predicting whether the subject will perform the undesired behavior or not perform the desired behavior; and
intervening when the risk analyzed is above a particular threshold.

2. The method according to claim 1, wherein the data received is extracted from a calendar.

3. The method according to claim 1, wherein the input source is an activity monitor configured to deliver data to a computing resource and the step of receiving a level of available self control includes calculating the level of available self control based on the data delivered.

4. The method according to claim 1, wherein the step of receiving a level of available self control includes receiving a food log or at least one image of food and determining the level of available self control based on the food log or at least one image received.

5. The method according to claim 1, wherein the step of intervening includes delivering a notification to the subject suggesting that the subject perform an action or refrain from performing an action to lower the risk to a value below the threshold.

6. A system for coaching a subject, comprising:
a processor; and
a memory storing instructions, executable by the processor, wherein the instructions when executed by the processor cause the system to:
receive data corresponding to at least one of a desired behavior of the subject or an undesired behavior of the subject;
receive a level of available self control of the subject via an input source;
calculate a level of required self control needed to perform the desired behavior or suppress the undesired behavior;
analyze a risk based on the level of available self control and the level of required self control, wherein the risk is associated with predicting whether the subject will perform the undesired behavior or not perform the desired behavior; and
intervene when the risk analyzed is above a particular threshold.

7. The system according to claim 6, wherein the data received is extracted from a calendar.

8. The system according to claim 6, wherein the input source is an activity monitor configured to deliver data to a computing resource and wherein the instructions when executed by the processor further cause the system to calculate the level of available self control based on the data delivered.

9. The system according to claim 6, wherein the step of receiving a level of available self control includes receiving a food log or at least one image of food and using the food log or at least one image of food to determine the level of available self control.

10. The system according to claim 6, wherein the step of intervening includes delivering a notification to the subject suggesting that the subject perform an action or refrain from performing an action to lower the risk to a value below the threshold.

11. An apparatus for coaching a subject, comprising:
a receiving unit configured to receive data corresponding to at least one of a desired behavior of a subject or an undesired behavior of a subject;
a calculating unit configured to calculate a level of available self control of the subject and a level of required self control needed to perform the desired behavior or suppress the undesired behavior;
a risk assessment unit configured to analyze a risk based on the level of available self control and the level of required self control, wherein the risk is associated with predicting whether the subject will perform the undesired behavior or not perform the desired behavior; and
an intervention unit configured to intervene when the risk analyzed by the risk assessment unit is above a particular threshold.

12. The apparatus according to claim 11, wherein the data received is extracted from a calendar.

13. The apparatus according to claim 11, wherein the input source is an activity monitor configured to deliver data to a computing resource and the calculating unit is further configured to calculate the level of available self control based on the data delivered.

14. The apparatus according to claim 11, wherein the calculating unit is further configured to receive a food log or at least one image of food and determine the level of available self control based on the food log or at least one image received.

15. The apparatus according to claim 11, wherein the intervention unit is further configured to deliver a notification to the subject suggesting that the subject perform an action or refrain from performing an action to lower the risk to a value below the threshold.

16. A non-transitory computer readable storage medium storing a program which, when executed by a computer, causes the computer to perform a method for coaching a subject, comprising:
receiving data corresponding to at least one of a desired behavior of the subject or an undesired behavior of the subject;
receiving a level of available self control of the subject via an input source;
calculating a level of required self control needed to perform the desired behavior or suppress the undesired behavior;
analyzing a risk based on the level of available self control and the level of required self control, wherein the risk is associated with predicting whether the subject will perform the undesired behavior or not perform the desired behavior; and
intervening when the risk analyzed is above a particular threshold.

17. The non-transitory computer readable storage medium according to claim 16, wherein the data receive is extracted from a calendar.

18. The non-transitory computer readable storage medium according to claim 16, wherein the input source is an activity monitor configured to deliver data to a computing resource and the step of receiving a level of available self control includes calculating the level of available self control based on the data delivered.

19. The non-transitory computer readable storage medium according to claim 16, wherein the step of receiving a level of available self control includes receiving a food log or at least one image of food and determining the level of available self control based on the food log or at least one image received.

20. The non-transitory computer readable storage medium according to claim 16, wherein the step of intervening includes delivering a notification to the subject suggesting that the subject perform an action or refrain from performing an action to lower the risk to a value below the threshold.
